(19) United States
(12) Patent Application Publication
(10) Pub. No.: US 2021/0325655 A1
(43) Pub. Date: Oct. 21, 2021

(54) STEPPED BIOLOGICAL CHIP AND GENE SEQUENCING DEVICE FOR TESTING THE SAME

(71) Applicant: MOONLIGHT (NANJING) INSTRUMENT CO., LTD., Jiangsu (CN)

(72) Inventors: Meiqun YU, Jiangsu (CN); Wei ZHOU, Jiangsu (CN)

(73) Assignee: MOONLIGHT (NANJING) INSTRUMENT CO., LTD., Jiangsu (CN)

(21) Appl. No.: 17/297,466

(22) PCT Filed: Jan. 3, 2020
(86) PCT No.: PCT/CN2020/070194

§ 371 Date: May 27, 2021

(30) Foreign Application Priority Data
Nov. 27, 2018 (CN) 201811427102.6

(51) Int. Cl. G02B 21/16; G01N 21/64

(52) U.S. Cl. CPC G02B 21/16; G01N 21/6486; G01N 21/6458

(57) ABSTRACT

A stepped biological chip and a gene sequencing device for testing same. The stepped biological chip comprises substrates and small small small small small small fluorescent balls carrying biological information arranged on the top of the substrates, and the heights of the center point of adjacent small small small small small small fluorescent balls from a substrate bottom edge A are incremented or decremented by equal value. The gene sequencing device comprises a chip placing platform, the stepped biological chip, and a microscope objective lens placed above the chip placing platform and an illumination light source irradiated on the stepped biological chip at a certain angle of incidence.

### BACKGROUND

### Technical Field

The present invention relates to the technical field of optical measurement, and particularly, to a stepped biological chip and a gene sequencing device for testing the same.

### Description of Related Art

Gene sequencing is a novel medical detection technology. It makes great sense and is of great interest in China and overseas. Cost-efficient and high-speed gene sequencing techniques have always been a research topic for many companies.

At present, industrialized gene sequencing devices are equipped with 20x microscope objective lenses with high NA (generally having a submicron resolution) to capture fluorescence signals. The prior art also adopts coaxial illumination and optical filters (with an optical density greater than 6) for preventing laser beams from entering the camera to improve the signal-to-noise ratio (S/N) of the devices, thereby implementing the imaging analysis of fluorescence signals. Therefore, the existing gene sequencing devices are very expensive and have strict requirements for components.

### SUMMARY

Objective: The present invention is intended to provide a stepped biological chip, which enables a gene sequencing device to acquire information through taking a plurality of images in a same field of view (FoV), wherein each image has one step depth. Assuming that the biological chip has N steps, N images will be taken in the same FoV (by moving along Z-axis), and the accurate position information of the images will be acquired according to the times of imaging. If the objective lens used in the device has a resolution of δ, the resolution can be improved to δ/N by using the stepped biological chip.

The present invention is further intended to provide a gene sequencing device for testing the aforementioned stepped biological chip. The device can generate high-resolution images by using a standard microscope objective lens instead of a high-resolution microscope objective lens, and no optical filter is needed in the device, thereby greatly reducing the cost of the gene sequencing device.

In order to solve the aforementioned technical problems, the present invention adopts the following technical scheme:

A stepped biological chip is provided, comprising substrates and small fluorescent balls positioned at top ends of the substrates and carrying biological information, wherein heights from centers of small fluorescent balls to bottom edges A of the substrates ascend or descend by a constant.

A plurality of adjacent small fluorescent balls are arranged stepwise to form a group, and a plurality of groups of small fluorescent balls arranged stepwise are sequentially arranged on the biological chip.

A gene sequencing device for testing the aforementioned stepped biological chip is provided, comprising a chip placing platform and a stepped biological chip placed on the platform, and further comprising a microscope objective lens positioned above the chip placing platform and a light source irradiating the stepped biological chip at a certain angle of incidence, wherein the stepped biological chip comprises substrates and small fluorescent balls positioned at top ends of the substrates and carrying biological information, wherein vertical heights from centers of small fluorescent balls to bottom edges A of the substrates ascend or descend by a constant.

In each group of small fluorescent balls arranged stepwise, the height difference between adjacent small fluorescent balls is greater than twice the focal depth of the microscope objective lens.

The angle between incident light from the light source and the biological chip is between 0° and 90°, and particularly the angle of incidence is greater than arctan(D/2L), wherein D is the diameter of the microscope objective lens, and L is the vertical distance from the objective lens to the biological chip.

The horizontal interval between adjacent small fluorescent balls is in submicron. The step depth (the vertical height difference between the centers of adjacent small fluorescent balls) is greater than twice the focal depth of the objective lens to avoid interaction between small fluorescent images of different step depths and ensure signals of only one depth are captured in each image. Related signals are recognized according to the step depth (i.e., by quickly recognizing a corresponding small fluorescent ball). Thus, high-resolution imaging is implemented with an objective lens having a low NA.

Compared with the prior art, the technical scheme of the present invention has the following benefits:

The gene sequencing device of the present invention adopts a standard microscope objective lens with a low magnification, the FoV of which is far greater than that of the conventional 20x objective lens, thereby improving the scanning speed of the device. By oblique illumination, background light formed by illumination light rays are prevented, which largely improves the S/N of the system, and eliminates the need for high-precision optical filters to block laser beams. The biological chip used in the gene sequencing device of the present invention adopts a stepped structure, realizing high-resolution imaging with objective lenses with low magnification and low NA.

### BRIEF DESCRIPTION OF THE DRAWINGS

FIG. 1 illustrates the schematic structure of a gene sequencing device according to the prior art.

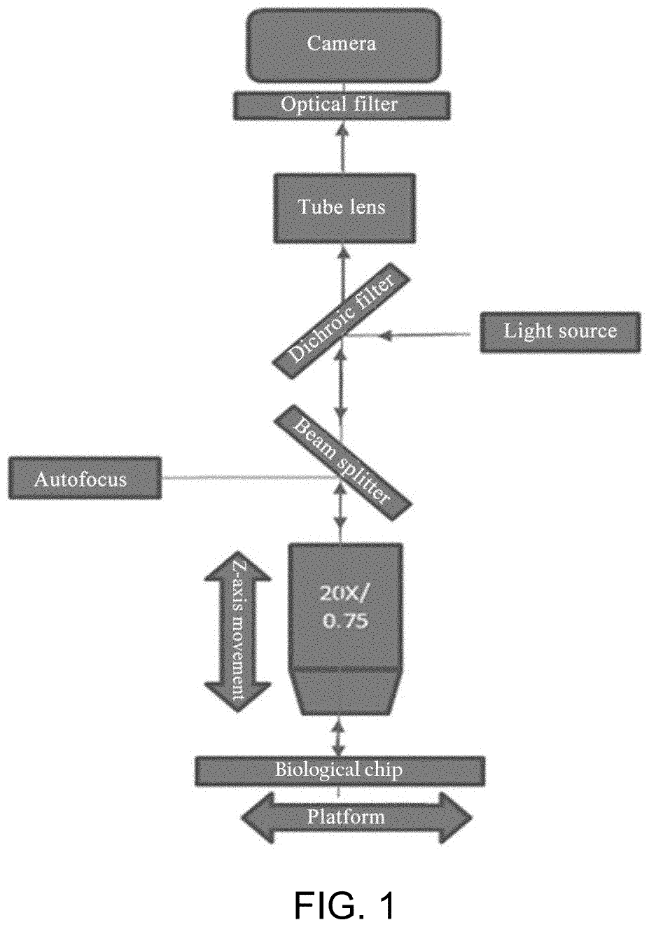

FIG. 2 illustrates the schematic structure of the gene sequencing device according to the present invention.

FIG. 3 illustrates the schematic structure of the stepped biological chip according to the present invention.

### DESCRIPTION OF THE EMBODIMENTS

The technical scheme of the present invention is further described below with reference to the drawings.

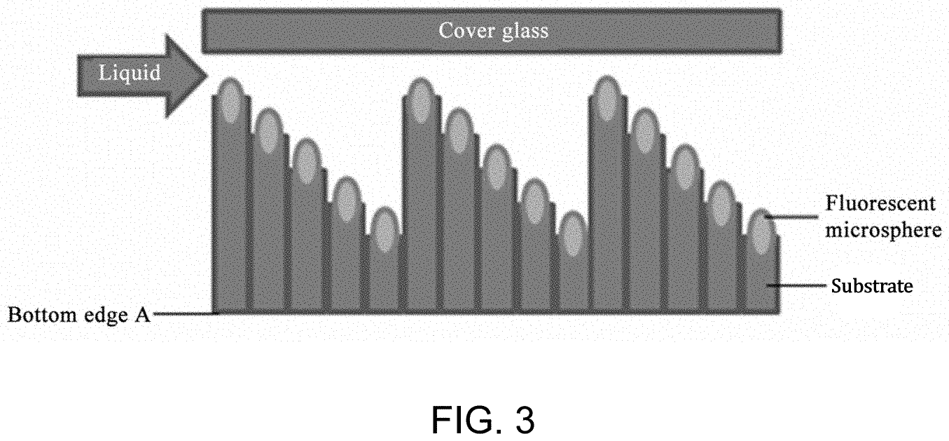

As shown in FIG. 3, the stepped biological chip of the present invention comprises substrates and small fluorescent balls fixed at top ends of the substrates and carrying biological information. The vertical heights from the centers of adjacent small fluorescent balls to bottom edges A of the substrates descend by a constant. Every five small fluorescent balls arranged stepwise form a group, and three such groups are sequentially arranged on the biological chip along the horizontal direction (three groups of small fluorescent balls are arranged in a horizontal line). In each group of small fluorescent balls arranged stepwise, the height difference between centers of adjacent small fluorescent balls is greater than twice the focal depth of the microscope objective lens.

The number of steps (the number of small fluorescent balls arranged stepwise in each group) and the step depth (the height difference between the centers of adjacent small fluorescent balls) is associated with the microscope objective lens used in the device. The microscope objective lens selected in this embodiment has a 5× magnification and an NA of 0.15. If a resolution of 0.9 μm (equivalent to a 20×/0.75 objective lens in conventional application) is required, 5 steps are needed since a 5× objective lens has a resolution of only 4.5 μm. That is, the number of small fluorescent balls arranged stepwise in each group is 5. In order to avoid interaction between images of two steps, the height difference between the two steps should be about 100 μm (twice the focal depth). Similarly, the testing speed of the gene sequencing device of the present invention will be greatly improved. The 5× objective lens has an object FoV of ø 5 mm (a standard objective lens with a field number of 25 mm), while the 20× objective lens used in conventional application has an object FoV of ø1.25 mm. Therefore, compared with the existing gene sequencing devices, the gene sequencing device of the present invention has a 16 times greater imaging area. Assuming that time consumed by chip moving, autofocusing and imaging remains constant and chips having the same area are detected, the gene sequencing device of the present invention has an 8 times faster testing speed.

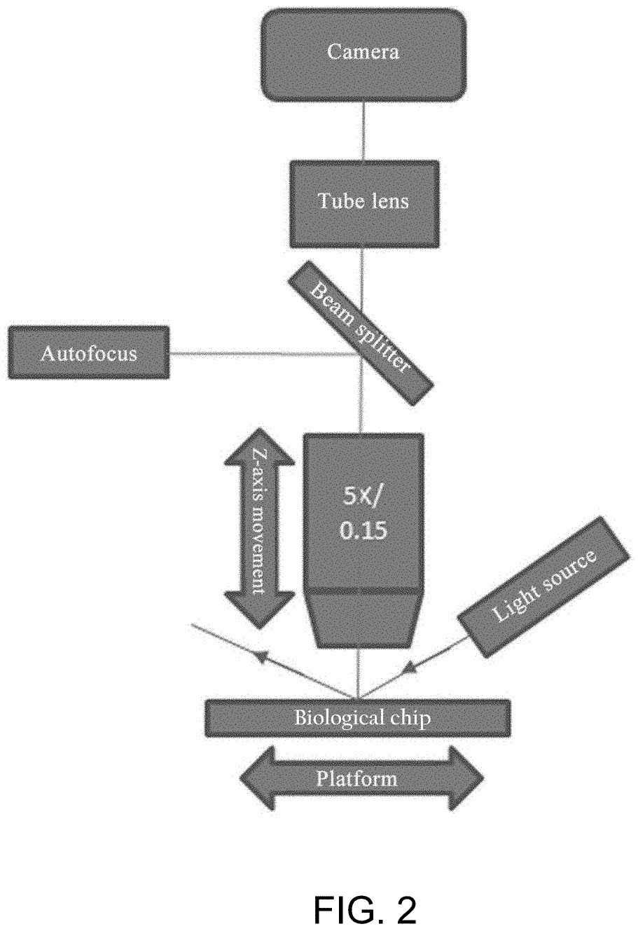

As shown in FIG. 2, the gene sequencing device of the present invention for testing the aforementioned stepped biological chip comprises a chip placing platform and the stepped biological chip placed on the platform, and further comprising a microscope objective lens positioned above the platform and a light source irradiating the stepped biological chip at a certain angle of incidence. The angle of incidence, i.e. the angle between incident light and the biological chip, is between 0° and 90°. Specifically, when the microscope objective lens in the device is selected, the angle of incidence is greater than arctan(D/2L), wherein D is the diameter of the microscope objective lens, and L is the vertical distance from the objective lens to the biological chip.

The gene sequencing device of the present invention adopts the oblique illumination. The light that is not converted into fluorescence cannot enter the imaging system to cause background noise through mirror reflection, and thus an optical filter for blocking the light source is not needed in the device. Compared with the conventional coaxial illumination, oblique illumination greatly reduces the noise of the system, and the optical filter for blocking the light source is no longer needed, thereby greatly reducing the cost. The angle of oblique incidence is related to the outer diameter and working distance of the chosen objective lens. Assuming that the diameter of the objective lens is D and the working distance (vertical distance from the objective lens to the biological chip) is L, the angle of oblique incidence (a certain angle between incident light and the biological chip) of the light source should be greater than arctan(D/2L).

The workflow of the conventional method is as follows: the light is emitted from a light source, enters the system through reflection of a dichroic filter, then reaches the biological chip through a beam splitter and an objective lens, and irradiates biological tissues to excite fluorescence. The fluorescence is collected by the objective lens and enters a camera through the beam splitter, the dichroic filter, a tube lens and an optical filter. A high-precision X/Y-axis moving platform is required due to the big size of biological chip and small FoV of the objective lens. Autofocusing is performed once after each movement of the platform to ensure that the biological chip is always on the focal plane of the objective lens. Because the energy of the light source is very high while the energy of the fluorescence is extremely weak, an optical filter with a very high optical density is required to block the light reflected by the objective lens, the beam splitter and the like.

The workflow of the device of the present invention is as follows: the light from the light source obliquely enters the biological chip, but is regularly reflected by the substrates and the cover glass and does not enter the fluorescence collection system. Therefore, both the optical filter and the dichroic filter can be removed to greatly reduce the cost. Similarly, the excited fluorescence is captured by the camera through the objective lens and the tube lens, and enters. The autofocus module is also used to ensure that the biological chip is always on the focal plane of the objective lens when the X/Y moving platform moves. A plurality of images will be taken in a same FoV, and a displacement in the Z-direction is required between two adjacent images, wherein the depth of the displacement is the depth difference between two adjacent steps. Because of the large FoV of the microscope objective lens, all information on a biological chip can be obtained in one FoV. The device of the present invention only takes a certain number of images for the biological chip along the Z-direction corresponding to the number of steps (i.e., one imaging for each step). In existing devices, a biological chip is moved several times in the XY-direction to give all information on the biological chip in a similar area, and focusing is performed once after each movement. Thus, the testing speed of the device of the present invention is much faster than that of existing devices.

1. A stepped biological chip, comprising substrates and small fluorescent balls positioned at top ends of the substrates and carrying biological information, wherein vertical heights from centers of the small fluorescent balls to bottom edges A of the substrates ascend or descend by a constant.

2. The stepped biological chip according to claim 1, wherein a plurality of adjacent small fluorescent balls are arranged stepwise to form a group, and multiple groups of small fluorescent balls arranged stepwise are sequentially arranged on the biological chip.

3. A gene sequencing device for testing the stepped biological chip according to claim 1, comprising a chip placing platform and a stepped biological chip placed on the chip placing platform, and further comprising a microscope objective lens positioned above the chip placing platform and a light source irradiating the stepped biological chip at a certain incidence angle, wherein the stepped biological chip comprises substrates and small fluorescent balls positioned at top ends of the substrates and carrying biological information, and vertical heights from centers of the small fluorescent balls to bottom edges A of the substrates ascend or descend by a constant.

4. The gene sequencing device according to claim 3, wherein in each group of the small fluorescent balls arranged stepwise, a height difference between the adjacent small fluorescent balls is greater than twice a focal depth of the microscope objective lens.

5. The gene sequencing device f according to claim 3, wherein an angle between an incident light from the light source and the stepped biological chip is between 0° and 90°.
